(19) United States
(12) Patent Application Publication
(10) Pub. No.: US 2019/0000550 A1
(43) Pub. Date: Jan. 3, 2019

(54) Ablation Catheter Having an Optical Fiber and an Adjustment Device

(71) Applicant: Vimecon GmbH, Herzogenrath (DE)

(72) Inventor: Kai Ulf Markus, Eschweiler (DE)

(21) Appl. No.: 16/062,901

(22) PCT Filed: Nov. 23, 2016
(86) PCT No.: PCT/EP2016/078581

§ 371 Date: Jun. 15, 2018

(30) Foreign Application Priority Data
Dec. 16, 2015 (DE) 10 2015 225 400.3

(51) Int. Cl. A61B 18/24

(52) U.S. Cl. CPC A61B 18/24; A61B 2018/00577

(57) ABSTRACT

An ablation catheter for ablating biological tissue, including an optical fiber extending through the catheter, and an out-coupling region for coupling out laser light into the external environment of the catheter, said catheter being provided with at least one adjustment device designed to bend the catheter at least in the area of the out-coupling region such that the out-coupling region can be aligned relative to the tissue to be ablated.

The invention relates to an ablation catheter for ablating biological tissue.

Ablation of biological tissue is performed with the aid of an electromagnetic wave, typically laser light, so as to heat the tissue to be ablated. Thereby, for instance, a faulty pulse propagation during excitation of the heart muscle tissue shall be avoided. In this regard, it is of particular importance to introduce the energy of the electromagnetic wave (laser light) into the tissue with maximum possible target accuracy, thus avoiding ablation of healthy tissue.

In conventional ablation catheters, it is difficult to achieve a precise targeting, relative to the tissue to be ablated, of the ablation region via which the electromagnetic wave is coupled from the catheter into the tissue.

It is an object of the invention to provide a catheter which allows for better-targeted ablation of biological tissue.

The catheter according to the invention is defined by the features of claim 1.

Thus, at least one adjustment device for actively bending the catheter is provided at least in the area of the out-coupling region so as to make it possible to adjust the out-coupling region relative to the tissue to be ablated and/or to adapt it to the shape of the tissue to be ablated. For this purpose, the catheter can be bent next to the out-coupling region, e.g. before the out-coupling region, behind the out-coupling region, proximally adjacent to the out-coupling region etc., or the out-coupling region itself can be bent. Thereby, it is rendered possible to advance the catheter in its not actively bent state until reaching the to-be-ablated region and there, with the aid of the adjustment device, to adjust the ablation region in a targeted manner to the to-be-ablated region of the tissue.

In this respect, various adjustment devices can be provided for bending the catheter in a respective plane. Typically, the out-coupling region for targeted emission of the laser light is formed substantially along a radiation direction. “Substantially” herein means that the radiation direction is a main radiation direction around which the laser light can be irradiated in a narrow angular range of a few degrees.

A first adjustment device can bend the catheter, along a circular arc having a predetermined first radius, in a plane arranged transversely to the radiation direction. Alternatively or additionally, a second adjustment device can bend the catheter, along a plane comprising the radiation direction, along a circular arc having a predetermined second radius, It is of particular advantage if at least one adjustment device is designed to bend the catheter in the area of the out-coupling region. Thus, the bent region of the catheter comprises the out-coupling region so that the out-coupling region can be adjusted in a targeted manner. In ablation catheters, as compared to other catheters, a special characteristic consists in that the out-coupling region will always move together with the rest of the catheter because the out-coupling region is fixedly connected to the optical fiber contained in the catheter. This effect shall be used for bending the out-coupling region and bringing it into a shape adapted to the to-be-ablated tissue, thus to achieve a uniformly continuous ablation by uniform contact between the out-coupling region and the tissue. Herein, the out-coupling region can be bent convexly and/or concavely.

Further, it is of particular advantage if the out-coupling region extends along the shortest possible length of the catheter, e.g. about 30 mm at maximum and particularly about 20 mm at maximum, e.g. by about 15 mm. Such a short length of the out-coupling region in combination with a targeted out-coupling of the laser light from the out-coupling region along a radiation direction allows for a well-aimed, spatially narrowly delimited ablation along a short line. Thereby, narrowly delimited regions of the tissue can be ablated in a well-specified manner. In conventional ablation catheters, typically, there are ablated lines corresponding to the length of the ablation region and partially having a length of several centimeters. Particularly in combination with a short out-coupling region for ablation along a short line, the use of the at least one adjustment device makes it possible to achieve a well-aimed, spatially narrowly delimited ablation.

Hereunder, exemplary embodiments of the invention will be explained in greater detail with reference to the Figures. The following is shown:

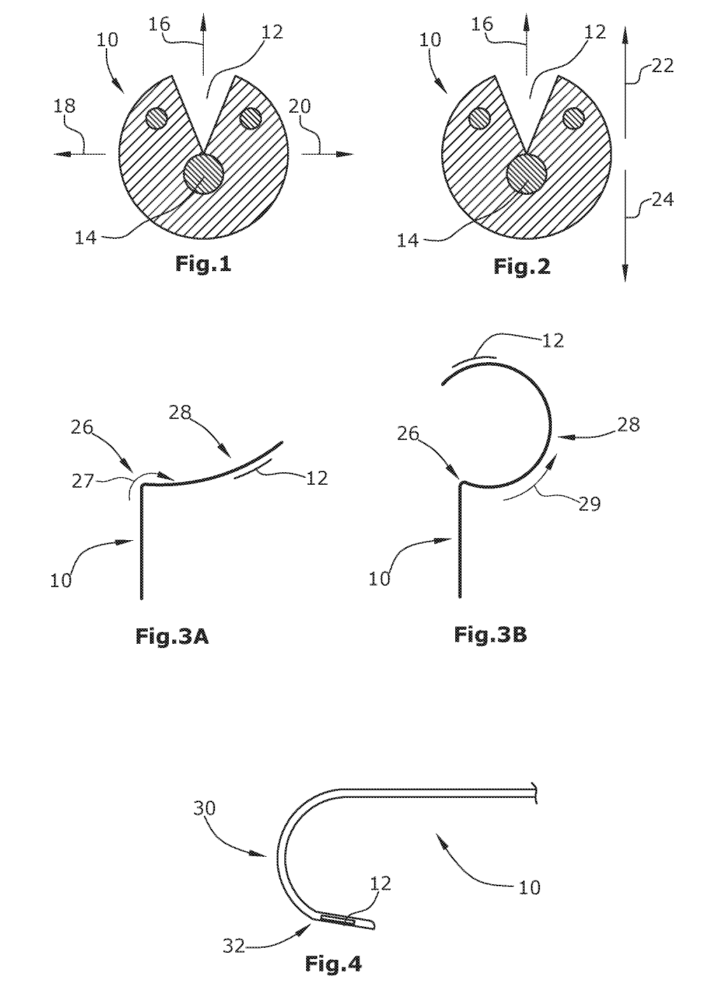

FIG. 1 is a cross-sectional view of a first exemplary embodiment,

FIG. 2 is a cross-sectional view of a second exemplary embodiment,

FIG. 3A is a view of a third exemplary embodiment in a first state,

FIG. 3B is a view of the exemplary embodiment according to FIG. 3A in a second state, and

FIG. 4 is a view of a fourth exemplary embodiment.

FIGS. 1 and 2 show cross-sectional views of the ablation catheter 10 in the area of the out-coupling region 12, In the out-coupling region 12, the laser light transported along the optical fiber 14 through the catheter 10 will be out-coupled from the catheter along the radiation direction 16.

In the exemplary embodiment according to FIG. 1, an adjustment device, not illustrated in the Figure, is designed to bend the catheter along the directions 18, 20 extending transversely to the decoupling direction 16. The catheter will thus be bend in the area of the out-coupling region 12. Thereby, the bending of the catheter is performed in a plane comprising the directions 18, 20 and extending transversely to the radiation direction 16.

The exemplary embodiment according to FIG. 2 is different from the one according to FIG. 1 in that the catheter is bendable in directions 22, 24 extending parallel to the radiation direction 16, which means, thus, in a plane comprising the directions 22, 24 and the radiation direction 16.

FIGS. 3A and 3B show an exemplary embodiment wherein the catheter 10 comprises two adjustment devices. A first adjustment device will bend the catheter, along arrow 27 in FIG. 3A, in a first region 26 (length portion of the catheter) arranged proximally behind the out-coupling region 12. A second adjustment device is designed to bend the catheter, along arrow 29 in FIG. 3B, in a second region 28, i.e. along a second length portion of the catheter that includes the out-coupling region 12. The bending with the aid of the first adjustment device in the region 26 is performed with a first radius, and the bending with the aid of the second adjustment device in the region 28 is performed with a second radius, wherein the first radius is distinctly smaller than the second radius. The first radius is so small that the catheter 10 forms a kinking site in the first region 26. The second radius of the second region 28 is distinctly larger.

In the first region 26, the catheter, while in the states shown in FIGS. 3A and B, is bent by about 90 degrees. In FIG. 3A, there does not yet take place an active bending of the catheter in the second region 28 with the aid of the second adjustment device. The catheter is flexible and can recede under the effect of external influences such as e.g. by contact with tissue. FIG. 3B shows the catheter in the curved state attained by means of the second adjustment device. The catheter has been bent in the second region 28 similar to a lasso or the letter C by more than zero degrees and preferably by more than 180 degrees but less than 360 degrees. It is of particular advantage if, in the exemplary embodiment in FIGS. 3A and 3B, the first adjustment device is designed to bend the catheter in the first region 26 by about 90 degrees, i.e. by circa 80 to 100 degrees, and in the second region 28 the catheter is bent with the aid of the second adjustment device by about 220 to about 320 degrees, e.g. by about 270 degrees (circa 270 degrees to 280 degrees). In all exemplary embodiments, the out-coupling region 12 is preferably arranged on the outer side of curvature that is being generated.

In the exemplary embodiment according to FIG. 4, the catheter 10 is bent, with the aid of a first adjustment device, by a first radius in a first region 30 and, with the aid of a second adjustment device, by a second radius in a second region 32. The planes of the curvatures in the regions 30, 32 can be arranged transversely relative to each other. The out-coupling region 12 is arranged distally of the regions 30, 32. In the exemplary embodiment according to FIG. 4, the first radius is distinctly larger than the second radius so that, in the first region 30, the bent catheter 10 describes an arc with continuous curvature and, in the second region 32, forms a kinking site distally of the first region 30 and proximally of the out-coupling region 12. In the first region 30, the catheter is bent by about 130 degrees and, in the second region 32, by about 45 degrees.

The adjustment devices can be designed to bend the catheter in a conventional manner, e.g. with the aid of pull wires extending within the catheter 10. Pulling on the proximal end of a pull wire will result in a one-sided eccentric stress on the catheter 10 which due to its flexibility will yield and undergo bending. Also other conventional variants can be conceived of, such as e.g. push wires which will be advanced within the catheter for bending it.

1. An ablation catheter for ablating biological tissue, comprising an optical fiber extending through the catheter for transport of laser light, and an out-coupling region for coupling out the laser light transported by the optical fiber into the external environment of the catheter,
wherein
the catheter is provided with at least one adjustment device designed to bend the catheter at least in a region of the out-coupling region in such a manner that the out-coupling region is aligned relative to the tissue to be ablated.

2. The device according to claim 1, wherein the adjustment device is configured to bend the catheter adjacent to at least one of the out-coupling region and the region of the out-coupling region.

3. The device according to claim 1, wherein a first adjustment device is provided for bending the catheter with a first radius and a second adjustment device is provided for bending the catheter with a second radius, the second radius being smaller than the first radius.

4. The device according to claim 3, wherein the second adjustment device is arranged between the first adjustment device and the out-coupling region.

5. The device according to claim 1, wherein the out-coupling region for targeted emission of the laser light is formed substantially along a decoupling direction, wherein a first adjustment device is provided for bending the catheter in a plane extending transversely to the decoupling direction and/or a second adjustment device is provided for bending the catheter in a plane comprising the decoupling direction.

6. The device according to claim 1, wherein the adjustment device is designed to bend the catheter with a constant radius by an angle of at least 180 degrees and less than 360 degrees.

7. The device according to claim 1, wherein a bending curvature comprises the out-coupling region, a catheter portion distally of the out-coupling region, and a catheter portion proximally of the decoupling region.

8. The device according to claim 1, wherein the out-coupling region extends along a length of the catheter of less than 30 mm.
